(19) United States
(12) Patent Application Publication
(10) Pub. No.: US 2020/0353064 A1
(43) Pub. Date: Nov. 12, 2020

(54) NOVEL MENINGOCOCCAL VACCINE COMPOSITION AND PROCESS THEREOF

(71) Applicant: MSD WELLCOME TRUST HILLEMAN LABORATORIES PVT. LTD., Delhi (IN)

(72) Inventors: Manoj kumar CHHIKARA, Delhi (IN); Kishore HARALE, Delhi (IN); Rakesh RANA, Delhi (IN); Davinder GILL, Delhi (IN)

(21) Appl. No.: 16/965,472

(22) PCT Filed: Jan. 29, 2019
(86) PCT No.: PCT/IN2019/050067

§ 371 Date: Jul. 28, 2020

(30) Foreign Application Priority Data
Jan. 29, 2018 (IN) 201811003233

(51) Int. Cl. A61K 39/095; A61K 47/64; A61K 47/61

(52) U.S. Cl. CPC A61K 39/095; A61K 47/6415; A61K 2039/6037; A61K 47/646; A61K 47/61

(57) ABSTRACT

The present invention is related to novel polysaccharide-protein conjugate vaccine formulation comprising of at least one of Neisseria meningitidis serogroup A, C, Y, W or X synthetic oligosaccharides (Men A, C, Y, W, X), each said oligosaccharide (OS) being conjugated separately to carrier protein, either none or at least one but not exceeding four bacterial capsular polysaccharide (PS) of Men A, C, Y, W or X, each said polysaccharide being conjugated separately to carrier protein, one or more buffer along with pharmaceutically acceptable components/excipients, with or without an adjuvant. The formulation is a mono- or bi- or multi-valent, liquid or lyophilized or a Liquid-Lyo combination formulation providing desired osmolality, desired pH, high stability and desired immunogenicity.

### FIELD OF THE INVENTION

The present invention relates to novel meningococcal vaccine composition and process to prepare thereof. More particularly, the present invention relates to the meningococcal conjugate vaccine composition of formulation in liquid or lyophilized form employing serogroups A, C, Y, W, X where at least one serogroup conjugate is based on synthetic oligosaccharides.

### BACKGROUND OF THE INVENTION

Meningococcal disease is an acute, potentially severe illness caused by the bacterium Neisseria meningitidis (N. meningitidis or Meningococcus). It has been mentioned on the official website of the WHO that N. meningitidis is one of the most common causes of bacterial meningitis in the world and the only bacterium capable of causing large epidemics of meningitis. Explosive epidemics with incidence rates of up to 1000 cases per 100,000 inhabitants have been reported, particularly in sub-Saharan Africa.

There are 13 serogroups of meningococcus namely, A, B, C, D, 29E, H, I, K, L, W135, X, Y and Z, out if which the six major disease-causing serogroups are serogroups A, B, C, W, X, and Y.

Out of these serogroups, capsular polysaccharides of some of them elicit suitable immune response while the others are poorly immunogenic due to chemical structure and therefore do not have the potential of mitigating or preventing the disease when developed as a conjugate vaccine.

Further, the conventional bacterial polysaccharide based conjugates display heterogeneity and sometimes there is presence of highly toxic components and other host cell impurities which are difficult to remove which also can interfere in achieving desired immune response. Organic synthesis can provide defined carbohydrate epitopes in high purity without host cell impurities and in relatively large amounts for controlled conjugation to a carrier protein. In such an approach, synthetic saccharides are equipped with an in-built artificial spacer/linker attached during the organic synthesis process in order to facilitate selective conjugation to a carrier protein.

Oligosaccharides (OS) which correspond to short fraction of natural bacterial capsular polysaccharides (PS) are recognized by antibodies raised against high molecular weight native polysaccharide antigens. The oligosaccharides give promising possibilities as lead vaccine candidates as they are not only immunogenic, but can also function as haptens in their protein conjugates that can elicit specific antibodies in animal models and in humans. Advances in the field of biological research and new generation organic synthetic vaccine technology have provided more effective chemical assembly of the complex oligosaccharide fragments in organic synthetic lab which are generally available on and are purified from the surface of pathogenic bacteria.

Given the fact that the synthetic oligosaccharide provides the effective lead compounds for the biological research, specifically in the field of vaccine technology, the significant research is going on for the preparation of the synthetic oligosaccharides and their protein conjugates. However, there is no general protocol for the preparation of the oligosaccharide of the biological importance. The chemical synthesis of each lead conjugate molecule is a research project which takes long and systematic experimentation. The affordability and availability of the synthetic conjugate vaccines is a significant problem which requires a process that enables the availability of synthetic conjugate vaccines in a time-effective and cost-effective manner.

Therefore, there is a need to provide a synthetic vaccine formulation which is cost-effective and efficacious in comparison to the fully conventional bacterial conjugate vaccine. To achieve the best efficacy it may so require addition of one or more conventional polysaccharide conjugates in the multivalent formulation of synthetic oligosaccharide conjugates, hence, alternatively, there is a need for hybrid vaccines which are combination of polysaccharide-protein conjugates of conventional and synthetic polysaccharides by utilizing advantages from both the options which are efficacious, cost-effective, have patient compliance, reproducible antigen production process and broader coverage of serogroups/strains and meet the standard specifications.

### OBJECT OF THE INVENTION

In order to obviate the drawbacks in the existing state of art, the main object of present invention is to provide a novel meningococcal conjugate vaccine composition.

Another object of the present invention is to provide a composition of a novel meningococcal serogroups A, C, Y, W, X conjugate vaccine formulation.

Yet another object of the present invention is to provide a novel meningococcal conjugate vaccine composition employing the oligo-/poly-saccharide-protein conjugates of synthetic oligosaccharide and/or conventional polysaccharides or recombinant polysaccharides.

Yet another object of the present invention is to provide a novel meningococcal conjugate vaccine composition of formulation in liquid and/or lyophilized form or a combination thereof.

Yet another object of the present invention is to provide novel meningococcal conjugate vaccine compositions which meet the desired standard specifications.

Yet another object of the present invention is to provide a process to obtain novel meningococcal conjugate vaccine composition which gives rise to desired immunogenicity.

### SUMMARY OF THE INVENTION

Accordingly, the present invention provides a novel mono-, bi- or multi-valent meningococcal conjugate vaccine composition for serogroups A, C, Y, W, X employing the polysaccharide-protein conjugates of synthetic oligosaccharide and/or conventional polysaccharides or recombinant polysaccharides.

The meningococcal vaccine composition of the present invention provides a formulation of polysaccharide-protein conjugates where saccharide components of the conjugates for the serogroups A, C, Y, W, X are synthetic oligosaccharides, or the formulation is a hybrid combination of conjugates comprising of at least one synthetic oligosaccharide conjugate in combination with the conventional bacterial polysaccharide conjugates and/or recombinant bacterial polysaccharide conjugates.

The chain lengths in the synthetic oligosaccharide is variable, preferably from trimer to hexadecamer corresponding to short part of the large bacterial capsular polysaccharides. The size of the conventional bacterial capsular polysaccharides used in conjugates for hybrid formulations is also variable, ranging between 10 kD to 300 kD.

The carrier protein of the conjugates is obtained from gram positive or gram-negative bacteria, preferably selected from tetanus toxoid, diphtheria toxoid (DT), nontoxic mutant of DT (CRM197), outer membrane protein, factor H binding protein, Cholera Toxin B.

The conjugates are obtained from the conjugation technologies available in the public domain such as thio-ether conjugation, reductive amination, cyanylation or carbamate chemistry.

In a preferred embodiment, the vaccine composition is a mono-, bi- or multi-valent meningococcal conjugate vaccine composition of formulation comprising of one or more meningococcal serogroups A, C, Y, W and X, where at least one conjugate is comprising of synthetic oligosaccharide preferably with an in-built linker and others are synthetic oligosaccharide conjugates or conventional polysaccharide conjugates.

The composition of the present invention may further comprise adjuvant that preferably belong to but not limited to aluminum adjuvants.

The composition also comprises pharmaceutically acceptable excipients and buffers including but not limited to phosphate buffer, Tris buffer, MES buffer, histidine buffer etc., sugars e.g. sucrose, trehalose, mannitol etc., and detergents like tween 80 etc.

The novel meningococcal vaccine composition of the present invention is in liquid or lyophilized form or a combination of liquid and lyophilized components. The composition meets the desired standard specifications and shows comparable immune response against respective serogroups to the fully conventional bacterial polysaccharide based licensed conjugate vaccine.

The present invention is to provide a process to obtain novel meningococcal conjugate vaccine composition.

### BRIEF DESCRIPTION OF DRAWINGS

FIG. 1 shows Post 3-dose mouse Anti-MenA IgG/Serum Bactericidal titers after immunization with different dosages (3 and 1 μg saccharide content per dose) of a mono-valent meningococcal conjugate formulation containing synthetic MenA tetramer-CRM conjugates in comparison to the vehicle control.

FIG. 2 shows Post 1, 2, 3-dose mouse Anti-MenC IgG titers after immunization with a mono-valent meningococcal conjugate formulation containing synthetic MenC tetramer-TT conjugates having 1 μg saccharide content per dose in six independent studies.

FIG. 3 shows Post 3-dose mouse Anti-MenC Serum Bactericidal titers after immunization with a mono-valent meningococcal conjugate formulation containing synthetic MenC tetramer-TT conjugates having 1 μg saccharide content per dose in comparison to anti-MenC titers against licensed MenACYW conjugate vaccine.

FIG. 4 shows Post 3-dose mouse Anti-MenC IgG and Serum Bactericidal titers after immunization with a mono-valent meningococcal conjugate formulation containing synthetic MenC octamer-TT conjugates having 1 μg saccharide content per dose in comparison to the response against licensed MenACYW conjugate vaccine.

FIG. 5 shows Post 3-dose mouse Anti-MenY IgG/Serum Bactericidal titers after immunization with different dosages (3, 1 and 0.3 μg saccharide content per dose) of a mono-valent meningococcal conjugate formulation containing synthetic MenY tetrmer-TT conjugates in comparison to the vehicle control.

FIG. 6 shows Post 3-dose mouse Anti-MenX IgG/Serum Bactericidal titers after immunization with different dosages (1 and 0.1 μg saccharide content per dose) of different mono-valent meningococcal conjugate formulations containing synthetic MenX tetramer-TT conjugates in comparison to vehicle control and non-conjugated MenX oligomer control.

FIG. 7 shows Post 3-dose mouse Anti-MenCYWX IgG/Serum Bactericidal titers after immunization with meningococcal conjugate formulations containing synthetic MenCYWX-CRM conjugates having 1 μg saccharide content per dose for each serogroup in comparison to licensed MenACYW conjugate vaccine.

FIG. 8 shows Post 3-dose mouse Anti-MenACYWX Serum Bactericidal titers after immunization with penta-valent meningococcal conjugate formulations containing synthetic MenCYWX-CRM conjugates and MenA PS-TT conjugates having 1 μg saccharide content per dose for each serogroup in comparison to licensed MenACYW conjugate vaccine and a vehicle control.

### DETAILED DESCRIPTION OF INVENTION WITH NON-LIMITING EXAMPLES AND ILLUSTRATIONS

Accordingly, the present invention provides a novel oligosaccharide and/or polysaccharide-protein conjugate vaccine composition and formulation thereof. More particularly, the present invention relates to a conjugate vaccine composition comprising of at least one synthetic oligosaccharide based-protein conjugate produced using conjugation chemistry. The composition of present invention is capable of being used in production of a monovalent, bivalent, trivalent, tetravalent and pentavalent meningococcal conjugate vaccine.

The novel vaccine formulation of present invention comprises of polysaccharide-protein conjugates along with pharmaceutically acceptable components/excipients. All the conjugates in vaccine composition and formulation have same or different carrier protein. The present invention provides a monovalent up to pentavalent meningococcal conjugate vaccine formulation.

The novel all synthetic oligosaccharide-protein conjugate vaccine composition and formulation of present invention comprises of either one oligosaccharide-protein conjugates of the five individual oligosaccharide-protein conjugates selected from meningococcal serogroups A, C, Y, W X conjugated with carrier protein or any one serogroup in combination with one or more of other oligosaccharide-protein conjugates. The oligosaccharides (oligomers or OS) are selected from those corresponding to gram negative bacteria Neisseria meningitidis serogroup A, C, Y, W and X capsular polysaccharides. The carrier protein used for preparing different conjugates is selected from but not limited to Tetanus Toxoid (TT) or non-toxic mutant of diphtheria toxin that is cross reacting material (CRM-197 or simply CRM). The novel hybrid vaccine formulation of said Men A, C, Y, W, X-TT/CRM conjugates are obtained employing optimized combination of at least one synthetic oligosaccharide-protein conjugates and one to four bacterial polysaccharide-protein conjugates. The synthetic oligosaccharide or polysaccharide of said MenC or MenW or MenY serogroups can be O-Acetylated or de-O-acetylated and preferably de-O-Acetylated.

Said at least one synthetic conjugate is conjugated to a carrier protein employing thio-ether chemistry. Either none or at least one but not exceeding four of said conjugates are derived using bacterial capsular polysaccharide corresponding to MenA, C, Y, W or X conjugated to a carrier protein employing either cyanylation chemistry or carbamate chemistry or a combination of both.

The oligomers are activated by addition of reactive thiol group suitable for conjugation with carrier protein having reactive maleimide group to obtain conjugates with high antigenicity and high immunogenicity. The oligomers having tetramer to octamer repeat units for different serogroups are activated and used for the conjugation with carrier protein but not limited to TT or CRM. The conjugates are produced having linker arm between oligomer and protein moities. The linker is attached to either oligomer or carrier protein or both the oligomer and protein.

In one of the preferred embodiments, the novel mono- or bi- or multi-valent oligosaccharide/polysaccharide-protein conjugate vaccine formulation of the present invention comprises of meningococcal serogroups A, C, Y, W, X oligomers prepared synthetically and each individually conjugated to tetanus toxoid (TT) or CRM-197 using optimized thioether chemistry. Each said conjugate has the protein to polysaccharide ratio between 0.15-1.2.

The novel mono- or bi- or multi-valent polysaccharide-protein conjugate vaccine formulation of the present invention comprises of at least one meningococcal serogroups A, C, Y, W, X conjugates using oligomers prepared by organic synthesis with either none or at least one but not exceeding four serogroup conjugates being prepared by fermentation based polysaccharides and each individually conjugated to tetanus toxoid or CRM-197 using optimized chemistries for each conjugate. Each said conjugate has the protein to polysaccharide ratio between 0.2-1.2.

The novel mono- or bi- or multi-valent polysaccharide-protein conjugate vaccine formulation of the present invention comprises of either fully synthetic or combination with one or more fermentation based meningococcal serogroups A, C, Y, W, X polysaccharides each individually conjugated to tetanus toxoid or CRM-197 mixed with one or more buffer and one or more pharmaceutically acceptable excipients, with or without adjuvant.

The pharmaceutically acceptable excipients can be adjuvant, buffer, preservative, stabilizer, surfactant, either alone or in combination. The formulation of present invention is a liquid or lyophilized formulation or a liquid-lyo combination with mono- or multi-dose regimen with or without a preservative.

The free saccharide limit for each of the serogroup oligosaccharide or polysaccharide in the novel formulation of the present invention is <20% and preferably <15% at the time of preparation of formulation.

Table 1 shows novel mono- or bi- or tri- or tetra- or penta-valent liquid oligosaccharide/polysaccharide-protein conjugate vaccine formulations of the present invention.

TABLE 1

| | Concentration | | | Adjuvant | |
|---|---|---|---|---|---|
| | of | | | (Aluminum | |
| Active | active | | | phosphate or | |
| Ingredient | ingredient | Buffer | Excipient | hydroxide) | Water |
| One or more | 4-20 μg | 5-30 mM | 0-150 mM NaCl | With or without | qs |
| Synthetic Men | saccharide/ | Phosphate | sufficient to | 250-2000 μg | |
| A, C, Y, W, X | serogroup/ml | buffered | maintain | Al⁺⁺⁺/ml | |
| oligomer each | | saline (pH | osmolality | | |
| conjugated to | | 7.0 ± 0.2) | between 240-330 | | |
| TT or CRM | | | mOsmol/Kg | | |
| | 4-20 μg | 5-30 mM | 5-30 mM | With or without | qs |
| | saccharide/ | Phosphate | Histidine and 0- | 250-2000 μg | |
| | serogroup/ml | buffered | 150 mM NaCl | Al⁺⁺⁺/ml | |
| | | saline (pH | sufficient to | | |
| | | 7.0 ± 0.2) | maintain | | |
| | | | osmolality | | |
| | | | between 240-330 | | |
| | | | mOsmol/Kg | | |
| | 4-20 μg | 5-30 mM | 0-150 mM NaCl | With or without | qs |
| | saccharide/ | Histidine | sufficient to | 250-2000 μg | |
| | serogroup/ml | | maintain | Al⁺⁺⁺/ml | |
| | | | osmolality | | |
| | | | between 240-330 | | |
| | | | mOsmol/Kg | | |
| One or more | 4-20 μg | 5-30 mM | 0-150 mM NaCl | With or without | qs |
| synthetic Men | saccharide/ | Phosphate | sufficient to | 250-2000 μg | |
| A, C, Y, W, X | serogroup/ml | buffered | maintain | Al⁺⁺⁺/ml | |
| oligomer | | saline (pH | osmolality | | |
| conjugated to | | 7.0 ± 0.2) | between 240-330 | | |
| TT or CRM and | | | mOsmol/Kg | | |
| combined with | | | | | |
| one to four | | | | | |
| bacterial | 4-20 μg | 5-30 mM | 5-30 mM | With or without | qs |
| polysaccharides | saccharide/ | Phosphate | Histidine and | 250-2000 μg | |
| belonging to | serogroup/ml | buffered | 0-150 mM NaCl | Al⁺⁺⁺/ml | |
| other | | saline (pH | sufficient to | | |
| serogroups each | | 7.0 ± 0.2) | maintain | | |
| conjugated to | | | osmolality | | |
| TT or CRM | | | between 240-330 | | |
| | | | mOsmol/Kg | | |
| | 4-20 μg | 5-30 mM | 0-150 mM NaCl | With or without | qs |
| | saccharide/ | Histidine | sufficient to | 250-2000 μg | |
| | serogroup/ml | | maintain | Al⁺⁺⁺/ml | |
| | | | osmolality | | |
| | | | between 240-330 | | |
| | | | mOsmol/Kg | | |

The ingredients are mixed by stirring at room temperature for 0.5-2 hours and followed by filling in vials and storage at 2-8° C.

Table 2 shows the novel mono- or bi- or tri- or tetra- or penta-valent lyophilized oligosaccharide/polysaccharide-protein conjugate vaccine formulation of the present invention.

TABLE 2

| | Concen- | | | | Adjuvant | |
|---|---|---|---|---|---|---|
| | tration | | | | (Aluminum | |
| | of | | | | or | |
| Active | active | | | | phosphate | Water |
| Ingredient | ingredient | Buffer | Excipient | Stabilizer | hydroxide) | (MQW) |
| Lyophilized | 4-20 μg | 5-30 mM | 0-150 mM | 0-4% of | With or | qs |
| one | saccha- | Phosphate | NaCl | Sucrose/ | without | |
| or more | ride/ | buffered | sufficient to | Maltose/ | 250- | |
| Synthetic | sero- | saline (pH | maintain | Mannitol/ | 2000 μg | |
| Men A, C, | group/ | 7.0 ± 0.2) | osmolality | Trehalose/ | Al⁺⁺⁺/ml | |
| Y, W, X | ml | | between | Lactose; 0- | | |
| oligomer | | | 240-330 | 20mM | | |
| each | | | mOsmol/ | Glycine | | |
| conjugated | | | Kg | | | |
| to TT | 4-20 μg | 5-30 mM | 5-30 mM | 0-4% of | With or | qs |
| or CRM | saccha- | Phosphate | Histidine | Sucrose/ | without | |
| with | ride/ | buffered | and 0-150 | Maltose/ | 250- | |
| moisture | sero- | saline (pH | mM NaCl | Mannitol/ | 2000 μg | |
| content <3% | group/ | 7.0 ± 0.2) | sufficient to | Trehalose/ | Al⁺⁺⁺/ml | |
| | ml | | maintain | Lactose;0- | | |
| | | | osmolality | 20mM | | |
| | | | between | Glycine | | |
| | | | 240-330 | | | |
| | | | mOsmol/ | | | |
| | | | Kg | | | |
| | 4-20 μg | 5-30 mM | 0-150 mM | 0-4% of | With or | qs |
| | saccha- | Histidine | NaCl | Sucrose/ | without | |
| | ride/ | | sufficient to | Maltose/ | 250- | |
| | sero- | | maintain | Mannitol/ | 2000 μg | |
| | group/ | | osmolality | Trehalose/ | Al⁺⁺⁺/ml | |
| | ml | | between | Lactose; 0- | | |
| | | | 240-330 | 20mM | | |
| | | | mOsmol/ | Glycine | | |
| | | | Kg | | | |
| Lyophilized | 4-20 μg | 5-30 mM | 0-150 mM | 0-4% of | With or | qs |
| One or more | saccha- | Phosphate | NaCl | Sucrose/ | without | |
| synthetic | ride/ | buffered | sufficient to | Maltose/ | 250- | |
| Men A, C, | sero- | saline (pH | maintain | Mannitol/ | 2000 μg | |
| Y, W, X | group/ | 7.0 ± 0.2) | osmolality | Trehalose/ | Al⁺⁺⁺/ml | |
| oligomer | ml | | between | Lactose; 0- | | |
| conjugated | | | 240-330 | 20mM | | |
| to TT or | | | mOsmol/ | Glycine | | |
| CRM and | | | Kg | | | |
| combined | 4-20 μg | 5-30 mM | 5-30 mM | 0-4% of | With or | qs |
| with one | saccha- | Phosphate | Histidine | Sucrose/ | without | |
| to four | ride/ | buffered | and 0-150 | Maltose/ | 250- | |
| bacterial | sero- | saline (pH | mM NaCl | Mannitol/ | 2000 μg | |
| polysac- | group/ | 7.0 ± 0.2) | sufficient to | Trehalose/ | Al⁺⁺⁺/ml | |
| charides | ml | | maintain | Lactose; 0- | | |
| each | | | osmolality | 20mM | | |
| conjugated | | | between | Glycine | | |
| to TT | | | 240-330 | | | |
| or CRM | | | mOsmol/Kg | | | |
| with | 4-20 μg | 5-30 mM | 0-150 mM | 0-4% of | With or | qs |
| moisture | saccha- | Histidine | NaCl | Sucrose/ | without | |
| content <3% | ride/ | | sufficient to | Maltose/ | 250- | |
| | sero- | | maintain | Mannitol/ | 2000 μg | |
| | group/ | | osmolality | Trehalose/ | Al⁺⁺⁺/ml | |
| | ml | | between | Lactose; 0- | | |
| | | | 240-330 | 20mM | | |
| | | | mOsmol/Kg | Glycine | | |

The lyophilized component of the vaccine formulation (Drug product) contains active ingredient with stabilizer with or without buffer with or without other excipients. The diluent component (for dissolving lyophilized active ingredients or lyophilized conjugates) of the vaccine formulation (Drug product) contains excipient and/or buffer with or without the adjuvant. The ingredients for lyophilized or diluent components are mixed by stirring at room temperature for 0.5-2 hours and followed by filling in vials and used for lyophilization or storage at 2-8° C.

Table 3 shows the novel mono- or bi- or tri- or tetra- or penta-valent lyophilized-liquid combination oligosaccharide/polysaccharide-protein conjugate vaccine formulation of the present invention.

TABLE 3

| | Concen- | | | | | |
|---|---|---|---|---|---|---|
| | tration | | | | Adjuvant | |
| | of | | | | (Aluminum | |
| | active | | | | phosphate | |
| Active | ingre- | | | | or | Water |
| Ingredient | dient | Buffer | Excipient | Stabilizer | hydroxide) | (MQW) |
| Lyophilized | 4-20 μg | 5-30 mM | 0-150 | 0-4% of | With or | |
| portion | saccha- | Phosphate | mM NaCl | Sucrose/ | without | qs |
| with at | ride/ | buffered | sufficient to | Maltose/ | 250- | |
| least one of | sero- | saline (pH | maintain | Mannitol/ | 2000 μg | |
| Synthetic | group/ | 7.0 ± 0.2) | osmolality | Trehalose/ | Al⁺⁺⁺/ml | |
| Men A, C, | ml | | between | Lactose; 0- | | |
| Y, W, X | | | 240-330 | 20mM | | |
| oligomer each | | | mOsmol/Kg | Glycine | | |
| conjugated to | 4-20 μg | 5-30 mM | 5-30 mM | 0-4% of | With or | qs |
| TT or CRM | saccha- | Phosphate | Histidine | Sucrose/ | without | |
| with moisture | ride/ | buffered | and 0-150 | Maltose/ | 250- | |
| content <3% | sero- | saline (pH | mM NaCl | Mannitol/ | 2000 μg | |
| and liquid | group/ | 7.0 ± 0.2) | sufficient to | Trehalose/ | Al⁺⁺⁺/ml | |
| portion | ml | | maintain | Lactose; 0- | | |
| having one | | | osmolality | 20mM | | |
| to four of | | | between | Glycine | | |
| Synthetic | | | 240-330 | | | |
| Men A, C, | | | mOsmol/Kg | | | |
| Y, W, X | 4-20 μg | 5-30 mM | 0-150 | 0-4% of | With or | qs |
| oligomer | saccha- | Histidine | mM NaCl | Sucrose/ | without | |
| belonging | ride/ | | sufficient to | Maltose/ | 250- | |
| to other | sero- | | maintain | Mannitol/ | 2000 μg | |
| serogroups | group/ | | osmolality | Trehalose/ | Al⁺⁺⁺/ml | |
| each | ml | | between | Lactose; 0- | | |
| conjugated to | | | 240-330 | 20mM | | |
| TT or CRM | | | mOsmol/Kg | Glycine | | |
| Lyophilized | 4-20 μg | 5-30 mM | 0-150 | 0-4% of | With or | qs |
| potion with at | saccha- | Phosphate | mM NaCl | Sucrose/ | without | |
| least one of | ride/ | buffered | sufficient to | Maltose/ | 250- | |
| synthetic or | sero- | saline (pH | maintain | Mannitol/ | 2000 μg | |
| conventional | group/ | 7.0 ± 0.2) | osmolality | Trehalose/ | Al⁺⁺⁺/ml | |
| Men A, | ml | | between | Lactose; 0- | | |
| C, Y, W, | | | 240-330 | 20mM | | |
| X oligomer | | | mOsmol/Kg | Glycine | | |
| or | 4-20 μg | 5-30 mM | 5-30 mM | 0-4% of | With or | qs |
| polysac- | saccha- | Phosphate | Histidine | Sucrose/ | without | |
| charide | ride/ | buffered | and 0-150 | Maltose/ | 250- | |
| each | sero- | saline (pH | mM NaCl | Mannitol/ | 2000 μg | |
| conjugated | group/ | 7.0 ± 0.2) | sufficient to | Trehalose/ | Al⁺⁺⁺/ml | |
| to TT | ml | | maintain | Lactose; 0- | | |
| or CRM | | | osmolality | 20mM | | |
| with moisture | | | between | Glycine | | |
| content <3% | | | 240-330 | | | |
| and liquid | | | mOsmol/Kg | | | |
| portion | 4-20 μg | 5-30 mM | 0-150 | 0-4% of | With or | qs |
| having one | saccha- | Histidine | mM NaCl | Sucrose/ | without | |
| to four of | ride/ | | sufficient to | Maltose/ | 250- | |
| Synthetic or | sero- | | maintain | Mannitol/ | 2000 μg | |
| conventional | group/ | | osmolality | Trehalose/ | Al⁺⁺⁺/ml | |
| Men A, C, | ml | | between | Lactose; 0- | | |
| Y, W, X | | | 240-330 | 20mM | | |
| oligomer or | | | mOsmol/Kg | Glycine | | |
| polysac- | | | | | | |
| charide | | | | | | |
| belonging | | | | | | |
| to other | | | | | | |
| serogroups | | | | | | |
| each | | | | | | |
| conjugated to | | | | | | |
| TT or CRM | | | | | | |

The lyophilized portion contains at least one conjugate with stabilizer, with or without buffer, and with or without excipients; whereas, the liquid portion contains excipient and/or buffer with or without the adjuvant with at least one conjugate not contained in the lyophilized portion. The ingredients for lyophilized portion or the diluent components are mixed by stirring at room temperature for 0.5-2 hours and followed by filling in vials and used for lyophilization or storage at 2-8° C.

The formulation of the present invention is in liquid or lyophilized form or a combination thereof. The present invention also provides the optimum dosage of each of the conjugates in the vaccine composition and formulation. The optimum dose is 5-10 μg of serogroup A and X saccharide and 5 μg of serogroup C, Y and W saccharide per intended human dose without adjuvant or with 500 μg Al⁺⁺⁺ in form of aluminum phosphate adjuvant per human dose.

Different monovalent Meningococcal liquid and lyophilized formulations have been prepared to establish the immunogenicity of the formulations in the presence of different excipients and buffers, with or without adjuvant.

Various monovalent liquid or lyophilized formulations of meningococcal serogroups A,C,Y,W or X conjugate formulations with different dosages ranging from 0.5 to 20 μg per serogroup per intended human dose have been prepared by mixing the antigen with the diluent/buffers with or without adjuvant.

Table 4 shows the liquid formulations with 0.5-15 μg per serogroup per intended human dose (0.1-3.0 μg per serogroup per mouse dose).

TABLE 4
Matrix for the formulation of liquid monovalent Meningococcal conjugate vaccines.

| | | | | Antigen/ |
|---|---|---|---|---|
| Formulation | | | Aluminum | mouse |
| Code | Bulk conjugate | Diluent | phosphate | (μg OS) |
| sMen1 | MenA Tetramer-CRM | Normal saline | − | 3.0 |
| | (OS-Pr Ratio 0.25-1.2) | 0.9% (pH 7.0 ± 0.5) | | |
| sMen2 | MenA Tetramer-CRM | Normal saline | − | 1.0 |
| | (OS-Pr Ratio 0.25-1.2) | 0.9% (pH 7.0 ± 0.5) | | |
| sMen3 | MenA Tetramer-TT | Normal saline | − | 1.0 |
| | (OS-Pr Ratio 0.25-1.2) | 0.9% (pH 7.0 ± 0.5) | | |
| sMen4 | MenC Tetramer-TT | Normal saline | − | 1.0 |
| | (OS-Pr Ratio 0.25-1.2) | 0.9% (pH 7.0 ± 0.5) | | |
| sMen5 | MenC Tetramer-TT | Normal saline | − | 0.5 |
| | (OS-Pr Ratio 0.25-1.2) | 0.95% (pH 7.0 ± 0.5) | | |
| sMen6 | MenC Tetramer-TT | Normal saline | − | 0.2 |
| | (OS-Pr Ratio 0.25-1.2) | 0.9% (pH 7.0 ± 0.5) | | |
| sMen7 | MenC Tetramer-TT | Normal saline | − | 0.1 |
| | (OS-Pr Ratio 0.25-1.2) | 0.9% (pH 7.0 ± 0.5) | | |
| sMen8 | MenC Tetramer-TT | Normal saline | − | 1.0 |
| | (OS-Pr Ratio 0.15-0.25) | 0.9% (pH 7.0 ±0.5) | | |
| sMen9 | MenC Tetramer-TT | Normal saline | − | 0.5 |
| | (OS-Pr Ratio 0.15-0.25) | 0.9% (pH 7.0 ± 0.5) | | |
| sMen10 | MenC Octamer-TT | Normal saline | − | 1.0 |
| | (OS-Pr Ratio 0.25-1.2) | 0.9% (pH 7.0 ± 0.5) | | |
| sMen11 | MenC Octamer-TT | Normal saline | − | 0.5 |
| | (OS-Pr Ratio 0.25-1.2) | 0.9% (pH 7.0 ± 0.5) | | |
| sMen12 | MenC Octamer-TT | Normal saline | − | 0.1 |
| | (OS-Pr Ratio 0.25-1.2) | 0.9% (pH 7.0 ± 0.5) | | |
| sMen13 | MenC Octamer-TT | Normal saline | − | 1.0 |
| | (OS-Pr Ratio 0.4-1.2) | 0.9% (pH 7.0 ± 0.5) | | |
| sMen14 | MenC Octamer- | Normal saline | + | 1.0 |
| | CRM (OS-Pr Ratio 0.4-1.2) | 0.45% (pH 7.0 ± 0.5) | | |
| sMen15 | MenC Octamer- | Normal saline | − | 1.0 |
| | CRM (OS-Pr Ratio 0.4-1.2) | 0.9% (pH 7.0 ± 0.5) | | |
| sMen16 | MenW Tetramer-TT | Normal saline | − | 3.0 |
| | (OS-Pr Ratio 0.20-1.2) | 0.9% (pH 7.0 ± 0.5) | | |
| sMen17 | MenW Tetramer-TT | Normal saline | − | 1.0 |
| | (OS-Pr Ratio 0.20-1.2) | 0.9% (pH 7.0 ± 0.5) | | |
| sMen18 | MenW Tetramer-TT | Normal saline | − | 0.3 |
| | (OS-Pr Ratio 0.20-1.2) | 0.9% (pH 7.0 ± 0.5) | | |
| sMen19 | MenX Tetramer-TT | Normal saline | − | 1.0 |
| | (OS-Pr Ratio 0.20-1.2) | 0.9% (pH 7.0 ± 0.5) | | |
| sMen20 | MenX Tetramer-TT | Normal saline | − | 0.1 |
| | (OS-Pr Ratio 0.20-1.2) | 0.9% (pH 7.0 ± 0.5) | | |
| sMen21 | MenY Tetramer-TT | Normal saline | − | 3.0 |
| | (OS-Pr Ratio 0.20-1.2) | 0.9% (pH 7.0 ± 0.5) | | |
| sMen22 | MenY Tetramer-TT | Normal saline | − | 1.0 |
| | (OS-Pr Ratio 0.20-1.2) | 0.9% (pH 7.0 ± 0.5) | | |
| sMen23 | MenY Tetramer-TT | Normal saline | − | 0.3 |
| | (OS-Pr Ratio 0.20-1.2) | 0.9% (pH 7.0 ± 0.5) | | |
| sMen24 | MenC Tetramer | Normal saline | − | 1.0 |
| | | 0.9% (pH 7.0 ± 0.5) | | |
| sMen25 | MenC Octamer | Normal saline | − | 1.0 |
| | | 0.9% (pH 7.0 ± 0.5) | | |
| sMen26 | MenX Tetramer | Normal saline | − | 1.0 |
| | | 0.9% (pH 7.0 ± 0.5) | | |

Further different bi- or tri- or tetra- or penta-valent Meningococcal conjugate vaccine formulations have been prepared.

The bi- or tri- or tetra- or penta-valent fully synthetic oligosaccharides or the hybrid combination of synthetic oligosaccharides (OS) and bacterial polysaccharide (PS) based liquid or lyophilized or a liquid lyo combination of Meningococcal conjugate formulations have been prepared with or without the addition of Aluminum phosphate to different dosages for TT or CRM conjugates ranging from 4-20 μg saccharide (OS or PS) per serogroup per intended human dose.

For example, the following matrix in Table 5 has been used to prepare the different liquid formulations with TT or CRM conjugates containing 5-10 μg OS/PS per serogroup per intended human dose (1-2 μg OS/PS per serogroup per mouse dose):

TABLE 5
Matrix for the preparation of liquid bi-valent and multi-valent
meningococcal conjugate vaccine formulations

| | | | | | | OS/PS |
|---|---|---|---|---|---|---|
| | | | | | | per |
| | | | | | | mouse |
| | | | | | | dose |
| | | | | Adjuvant | | (Intended |
| | | | | (Alumi- | | human |
| Formu- | | | | num | | dose) |
| lation | | | | Phos- | | per |
| Code | Serogroup | PBS | NaCl | phate) | MQW | serogroup |
| sMen27 | Synthetic bi- | 10 mM | Normal | — | qs | l μg |
| | valent | | saline | | | (5 μg) |
| | (MenC and X | | 0.9% | | | each |
| | Tetramer- | | (pH 7.0 ± | | | |
| | TT) | | 0.5) | | | |
| sMen28 | Hybrid | 10 mM | Normal | — | qs | 1 μg |
| | tri-valent | | saline | | | (5 pg) |
| | (MenX | | 0.9% | | | each |
| | Tetramer- | | (pH 7.0 ± | | | |
| | TT and | | 0.5) | | | |
| | MenA and | | | | | |
| | C PS-TT | | | | | |
| | conjugate) | | | | | |
| sMen29 | Synthetic | 10 mM | Normal | — | qs | 1 μg |
| | tetravalent | | saline | | | (5 μg) |
| | (MenC, Y, | | 0.9% | | | each |
| | W, X-TT | | (pH 7.0 ± | | | |
| | conjugates) | | 0.5) | | | |
| sMen30 | Hybrid | 10 mM | Normal | — | qs | 1 μg |
| | pentavalent | | saline | | | (5 μg) |
| | (Licensed | | 0.9% | | | each |
| | MenACYW | | (pH 7.0 ± | | | |
| | Vaccine + | | 0.5) | | | |
| | MenX | | | | | |
| | Tetramer- | | | | | |
| | TT) | | | | | |
| sMen31 | Hybrid | 10 mM | Normal | — | qs | 1 μg |
| | Pentavalent | | saline | | | (5 μg) |
| | (MenWY | | 0.9% | | | each |
| | Tetramer- | | (pH 7.0 ± | | | |
| | TT MenA, | | 0.5) | | | |
| | C and X PS- | | | | | |
| | TT | | | | | |
| | conjugate) | | | | | |
| sMen32 | Hybrid | 10 mM | Normal | — | qs | 1 μg |
| | Pentavalent | | saline | | | (5 μg) |
| | (MenC | | 0.9% | | | each |
| | Tetramer- | | (pH 7.0 ± | | | |
| | CRM + | | 0.5) | | | |
| | MenA, | | | | | |
| | Y, W and | | | | | |
| | X PS-TT | | | | | |
| | conjugate) | | | | | |
| sMen33 | Hybrid | 10 mM | Normal | 1000 μg | qs | 1 μg |
| | pentavalent | | saline | Al⁺⁺⁺/ml | | (5 μg) |
| | (MenA PS- | | 0.45% | | | each |
| | TT and | | (pH 7.0 ± | | | |
| | MenC, Y, | | 0.5) | | | |
| | W, X- | | | | | |
| | CRM | | | | | |
| | conjugates) | | | | | |
| sMen34 | Hybrid | 10 mM | Normal | 1000 μg | qs | 1 μg |
| | pentavalent | | saline | Al⁺⁺⁺/ml | | (5 μg) |
| | (MenA | | 0.45% | | | each |
| | PS-TT and | | (pH 7.0 ± | | | MenC,Y, |
| | MenC, Y, | | 0.5) | | | W, X and |
| | W, X-CRM | | | | | 2 μg |
| | conjugates) | | | | | (10 μg) |
| | | | | | | MenA |
| sMen35 | Synthetic | 10 mM | Normal | — | qs | 1 μg |
| | pentavalent | | saline | | | (5 μg) |
| | (MenA, | | 0.9% | | | each |
| | C, Y, W, | | (pH 7.0 ± | | | |
| | X-CRM | | 0.5) | | | |
| | conjugates) | | | | | |
| sMen36 | Synthetic | 10 mM | Normal | — | qs | 1 μg |
| | pentavalent | | saline | | | (5 μg) |
| | (MenA, | | 0.9% | | | each |
| | C, Y, W, | | (pH 7.0 ± | | | |
| | X-TT | | 0.5) | | | |
| | conjugates) | | | | | |
| sMen37 | Synthetic | 10 mM | Normal | 1000 μg | qs | 1 μg |
| | pentavalent | | saline | Al⁺⁺⁺/ml | | (5 μg) |
| | (MenA, | | 0.45% | | | each |
| | C, Y, W, | | (pH 7.0 ± | | | |
| | X-CRM | | 0.5) | | | |
| | conjugates) | | | | | |
| sMen38 | Synthetic | 10 mM | Normal | 1000 μg | qs | 1 μg |
| | pentavalent | | saline | Al⁺⁺⁺/ml | | (5 μg) |
| | (MenA- | | 0.45% | | | each |
| | TT and | | (pH 7.0 ± | | | |
| | MenC, Y, | | 0.5) | | | |
| | W, X-CRM | | | | | |
| | conjugates) | | | | | |

The immunogenicity and antigenicity of the formulation of present invention is described by way of non-limiting examples.

### Example 1: Immunization of Mice with the Liquid Meningococcal Conjugate Vaccine Formulations

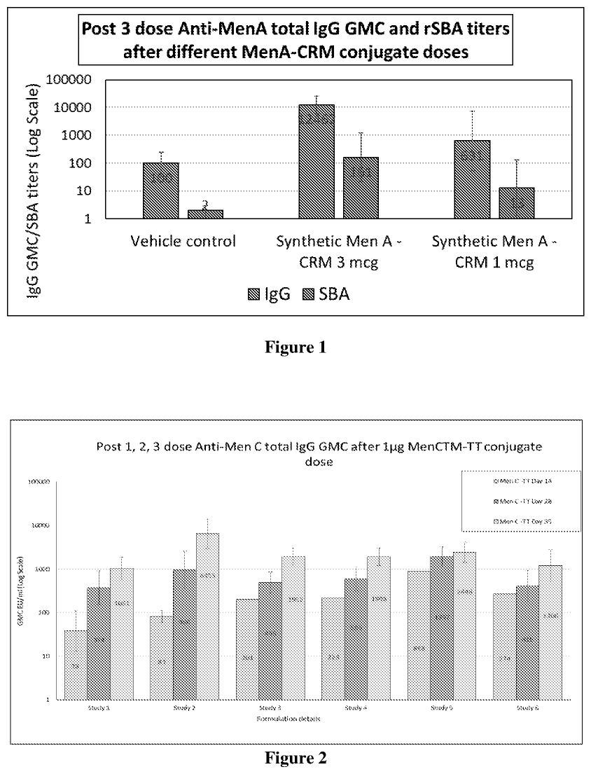

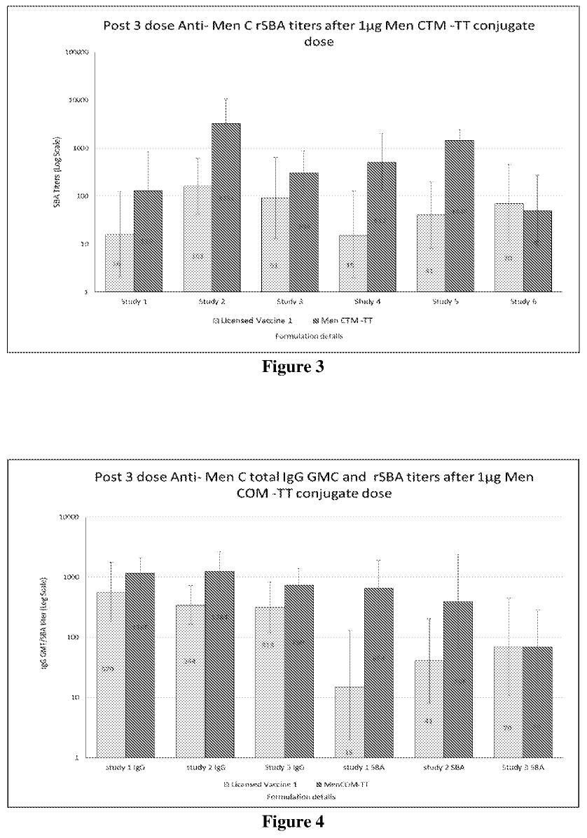

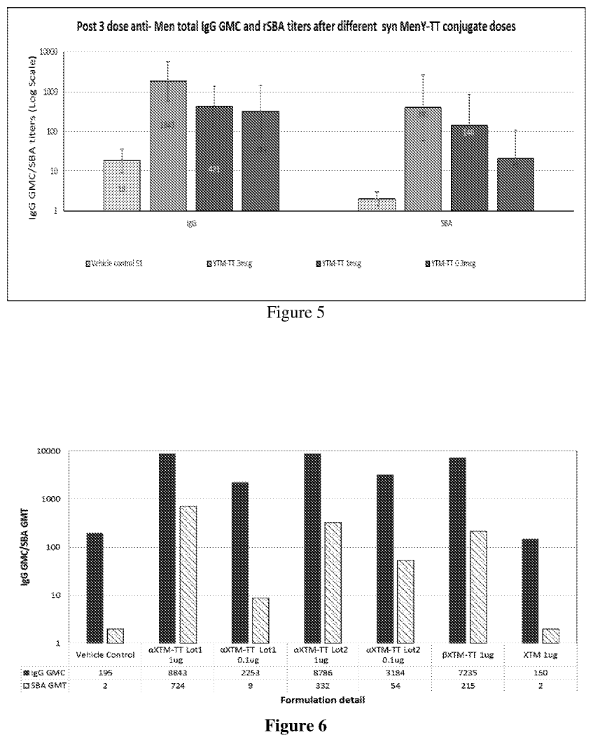

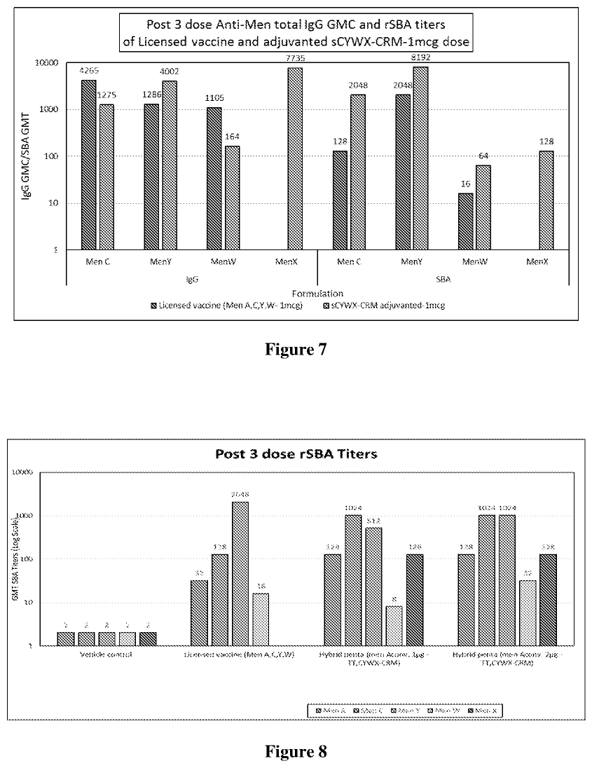

Groups of 6-8 female mice (6-9 weeks old) have been immunized at 2 week interval with either novel liquid adjuvanted or non-adjuvanted mono-valent or bi-valent or multi-valent meningococcal conjugate vaccine containing conjugates belonging to serogroup A, C, Y, W and/or X and conjugated to TT or CRM, a non-conjugated oligomer control, a vehicle control without bulk conjugates or a licensed ACYW conjugate vaccine. All immunizations have been performed by administering of vaccine via subcutaneous route in mice. Each mouse has been immunized with formulation equivalent to 0.1-3.0 μg oligosaccharide/polysaccharide per serogroup that is ⅕^{th} of intended human dose. Serogroup specific anti-meningococcal IgG antibody titers have been estimated by indirect ELISA and functional antibody titers by serum bactericidal assay in sera collected post 1, 2 and/or 3 dose. The post 1, 2 and 3 dose results for novel liquid mono-, bi- or multi-valent meningococcal ACYWX conjugate vaccines indicate booster responses and significantly high immunogenicity titers as compared to vehicle control, non-conjugated oligomers and non-inferior titers to the licensed vaccine IgG and SBA titers in both animal models (FIG. 1-8).

### Example 2: Determination of Anti-Meningococcal Polysaccharide Serogroup Specific IgG Titers by Indirect ELISA

Ninety six-well plates (Nunc Maxisorp) have been coated with serogroup specific standard Meningococcal PS by adding 100 μl per well mixture of a 5 μg/ml PS and m-HSA in PBS buffer, pH 7.3±0.1. Plates have been incubated overnight at 4° C., and then washed three times with PBS buffer (0.1% Brij 35 in PBS, pH 7.3±0.1) and blocked with 200 μl per well of 5% FBS solution in PBS buffer (0.1% Brij 35 in PBS, pH 7.3±0.1) for 1 hour at 37° C. Each incubation step has been followed by three PBS buffer wash. Reference and test sera samples have been diluted in PBS buffer (0.1% Brij 35, 5% FBS in PBS, pH 7.3±0.1), transferred into coated-blocked plates (200 μl), and serially two-fold diluted followed by overnight incubation at 4° C. Then 100 μl per well of optimally diluted peroxidase conjugated anti-mouse/rabbit IgG have been added and left for 1 hour at 25° C. The 100 μl per well of substrate, 3, 3′, 5, 5′-tetramethylbenzidine-H₂O₂ has been added for color development. After 10 minutes of development at 25° C., reaction has been stopped by adding 50 μl of 2 M H₂SO₄, and OD has been measured at 450 nm on Micro plate reader. Anti-Meningococcal serogroup polysaccharide IgG concentrations (in terms of ELISA Units/ml) for each formulation have been evaluated using Combistat software and the geometric mean concentrations (IgG GMC) have been shown for representative studies and formulation comparisons in FIG. 1, 2, 4-7.

### Example 3: Serum Bactericidal Assay (SBA) for the Serogroup Specific Functional Antibody Titration

N. meningitidis serogroup specific bacterial stock has been grown overnight on sheep blood agar plate at 37° C. with 5% CO₂. Isolated colonies have been picked and incubated on the surface of another sheep blood agar plate at 37° C. with 5% CO₂. The bacterial growth from second plate have been suspended in optimized SBA buffer for respective serogroup. The optical density (OD₆₅₀) of the suspension has been adjusted in working bacterial stock to achieve a colony count of 60-250 per spot in the end of the assay. Quality control (QC) sera and test sera samples have been heat inactivated for 30 min at 56° C. In micro well plate, 20 μl of serial two-fold dilutions of test serum has been mixed with 10 μl of bacteria at the working dilution and 10 μl of baby rabbit complement. For negative controls, bacteria have been incubated, in a separate well, with active baby rabbit complement without the test serum and with test serum and heat-inactivated baby rabbit complement. The well contents have been mixed by gently tapping the assay plate and incubated the plates for 1 hour at 37° C. with 5% CO₂. Ten μL sample from each well plated on blood agar plate by streak plate method. The blood agar plates have been incubated overnight at 37° C. with 5% CO₂ and colonies have been counted. The highest serum dilution showing ≥50% decrease in colony-forming units after incubation of bacteria with reaction mixture, as compared to respective active complement control has been considered as the SBA titer. The results for representative studies and formulation comparisons are presented in FIG. 1, 3-8.

1. A saccharide-protein conjugate vaccine formulation comprising:
at least one of Neisseria meningitidis serogroup A, C, Y, W or X synthetic oligosaccharide (Men A, C, Y, W, or X), each said oligosaccharide (OS) being conjugated separately to a carrier protein, said carrier protein being tetanus toxoid (TT) or a non-toxic mutant of diphtheria toxin that is cross reacting material (CRM) to obtain Men A, C, Y, W, or X-TT/CRM conjugates,
optionally, one to four bacterial capsular polysaccharides (PS) of Men A, C, Y, W or X each said polysaccharide being conjugated separately to a carrier protein, said carrier protein being TT or CRM to obtain Men A, C, Y, W, or X PS-TT/CRM conjugates,
one or more buffers along with pharmaceutically acceptable components/excipients, and
optionally, an adjuvant,
wherein said formulation is mono- or multi-valent, fully liquid, lyophilized or a liquid-lyo combination formulation.

2. The vaccine formulation as claimed in claim 1 wherein said formulation is a liquid formulation comprising:

| Ingredient | Quantity/Concentration |
|---|---|
| Men A, C, Y, W, or X-TT/CRM | 4-20 μg OS/PS per serogroup per ml |
| Buffer | 5-30 mM Phosphate buffered saline |
| Excipient | 0-150 mM NaCl |
| | 5-30 mM Histidine |
| Adjuvant: | Aluminum phosphate as |
| | 250-2000 μg Al⁺⁺⁺/ml |
| Water | qs |

3. The vaccine formulation as claimed in claim 1 wherein said formulation is a formulation comprising:

| Ingredient | Quantity/Concentration |
|---|---|
| Men A, C, Y, W, or X-TT/CRM | 10-20 μg OS/PS per serogroup per ml |
| Buffer | 10-25 mM Phosphate buffered saline |
| | (pH 7.0 ± 0.2) |
| Excipient | 0-150 mM NaCl |
| Adjuvant: | Aluminum phosphate as |
| | 750-1500 μg Al⁺⁺⁺/ml |
| Water | qs |

4. The vaccine formulation as claimed in claim 1 wherein said formulation is a formulation comprising:

| Ingredient | Quantity/Concentration |
|---|---|
| Men A, C, Y, W, or X-TT/CRM | 10-20 μg OS/PS per serogroup per ml |
| Buffer: | 10 mM PBS (pH 7.0 ± 0.2) |
| Excipient: | 0-150 mM NaCl |
| Adjuvant | Aluminum phosphate as |
| | 1000 μg Al⁺⁺⁺/ml |
| Water | qs |

5. The vaccine formulation as claimed in claim 1 wherein said formulation is a formulation comprising:

| Ingredient | Quantity/Concentration |
|---|---|
| Men A, C, Y, W, or X-TT/CRM | 10 μg OS/PS per serogroup per ml |
| Buffer: | 10 mM PBS (pH 7.0 ± 0.2) |
| Excipients: | 0-150 mM NaCl |
| Water | qs |

6. The vaccine formulation as claimed in claim 1 wherein said lyophilized formulation is a lyophilized formulation comprising:

| Ingredient | Quantity/Concentration |
|---|---|
| Men A, C, Y, W, or X-TT/CRM | 10-20 μg OS/PS per serogroup per ml |
| Buffer: | 5-20 mM PBS (pH 6.5-7.5) |
| Excipients: | 2-10% |
| Diluent | qs |

7. The vaccine formulation as claimed in claim 1 wherein said formulation is a liquid-lyo combination formulation comprising:

| Ingredient | Quantity/Concentration |
|---|---|
| Men A, C, Y, W, or X-TT/CRM | 10-20 μg OS/PS per serogroup per ml |
| Buffer: | 5-20 mM PBS (pH 6.5-7.5) |
| Excipients: | 2-10% |
| Diluent | qs |

8. (canceled)

9. (canceled)

10. (canceled)

11. (canceled)

12. (canceled)

13. The vaccine formulation as claimed in claim 1 wherein each said synthetic oligosaccharide is a trimer to hexadecamer corresponding to respective serogroup capsular polysaccharide and the capsular polysaccharide used in conjugation is degraded in the size range of 10-300 kDa.

14. The vaccine formulation as claimed in claim 1 wherein said conjugates comprise a linker arm between said oligosaccharide/polysaccharide and said carrier protein.

15. The vaccine formulation as claimed in claim 14 wherein said linker arm comprises a maleimide linker connected to the carrier protein attached to a thiolated linker of the oligosaccharide.

16. The vaccine formulation as claimed in claim 1 wherein said MenC, MenW and MenY oligomers are O-Acetylated or de-O-acetylated.

17. The vaccine formulation as claimed in claim 1 wherein each said conjugate has a carrier protein to polysaccharide ratio of 0.15-1.2.

18. The vaccine formulation as claimed in claim 1 wherein said pharmaceutically acceptable excipients are selected from preservative, stabilizer, surfactant, and combinations thereof.

19. A vial comprising the vaccine formulation as claimed in claim 6 wherein said excipient is selected from Sucrose, Maltose, Arginine, Lactose, Sorbitol, Mannitol, Trehalose, Histidine, Glycine, and combinations thereof.

20. The vaccine formulation as claimed in claim 6 wherein said diluent is selected from water, 5-20 mM phosphate buffered saline, aluminum phosphate as 250-1500 μg Al⁺⁺⁺/ml, and combinations thereof.

21. The vaccine formulation as claimed in claim 7 wherein a lyophilized portion comprises MenA-TT conjugate, MenA-CRM conjugate, MenC-TT conjugate, MenC-CRM conjugate, or combinations thereof.

22. The vaccine formulation as claimed in claim 7 wherein said excipient is selected from Sucrose, Maltose, Arginine, Lactose, Sorbitol, Histidine, Glycine, and combinations thereof.

23. (canceled)

24. (canceled)

25. (canceled)

26. (canceled)

27. The vaccine formulation as claimed in claim 1 wherein an osmolality of the formulation is 240-330 mOsmol/Kg.

28. The vaccine formulation as claimed in claim 1 wherein a pH of the formulation is 6.5-7.5.

29. The vaccine formulation as claimed in claim 1 wherein a lyophilized portion of the lyophilized or liquid-lyo formulation has a moisture content not more than 3%.

30. (canceled)

31. (canceled)

32. (canceled)
